(19) United States
(12) Patent Application Publication
(10) Pub. No.: US 2018/0280560 A1
(43) Pub. Date: Oct. 4, 2018

(54) SANITARY COMPOSITION WITH AN ACTIVE INGREDIENT FOR LOWERING THE pH OF THE VAGINAL ENVIRONMENT AND/OR THE EXTERNAL GENITALIA

(71) Applicant: FIDE s.r.o., Brtnice (CZ)

(72) Inventors: Petr RYSAVKA, Lulec (CZ); Libor Madera, Batelov (CZ)

(73) Assignee: FIDE s.r.o., Brtnice (CZ)

(21) Appl. No.: 15/478,267

(22) Filed: Apr. 4, 2017

(51) Int. Cl. A61L 15/20; A61L 15/36; A61L 15/28; A61L 15/42; A61F 13/472; A61F 13/49; A61F 13/20; A61F 13/84

(52) U.S. Cl. CPC A61L 15/20; A61L 15/36; A61L 15/28; A61L 15/42; A61F 2013/53908; A61F 13/49; A61F 13/20; A61F 13/8405; A61F 13/472

(57) ABSTRACT

A sanitary product with an active ingredient for reduction of the pH value of vaginal environment and/or external genitals, where the active ingredient contains crystalline lactic acid or salts thereof, and is applied on the surface layer of the sanitary product designed for contact with body and/or in the internal structure of the sanitary product. Lactic acid and/or salts thereof are contained in the active ingredient in the form of monoglyceryl monoester coating for sustained release of lactic acid from the sanitary product. Furthermore, the active ingredient contains the probiotic culture from the Lactobacillus, Bifidobacterium, or Streptococcus family, and prebiotic component from the group consisting of inulin or fructooligosaccharides.

### FIELD OF THE INVENTION

The invention relates to an area of intimate hygiene, more particular to a sanitary product with an active ingredient for reduction of the pH value of vaginal environment and/or external genitals.

### BACKGROUND OF THE INVENTION

The female urogenital tract is colonized by a complex microbial eco-system containing over 50 different bacterial species, and it is also characterized by specifically reduced pH value to limit the growth of undesirable microorganisms. For women at their reproductive age, the prevailing microorganisms are lactic acid producing bacteria from Lactobacillus family. Acidic pH value is important for preserving of healthy microbial flora across the vaginal area, where it has its natural antagonistic effect on pathogenic micro-organisms such as yeasts from Candida family, and undesirable bacterial species. The physiological pH value of the vaginal mucosa and external genitals is substantially lower, whereby the colonization of the vaginal mucosa by pathogenic microorganisms is effectively reduced. A common soap and alkaline intimate hygiene products, which is absolutely inappropriate for the external genitals, should never be used for the intimate female hygiene because the vaginal pH value is highly acid and the protective barrier is corrupted if an alkaline soap is used. In addition, perfumed soaps, sanitary slips, and other intimate hygiene products may irritate the sensitive skins and mucosa. Contrary to this, the intimate hygiene products with the pH value 5.5 are suitable.

The microbial eco-system and physiological pH of the external genitals and the vaginal mucosa of a healthy woman may be compromised by use of antibiotics, chemotherapeutics, antimycotics, during hormonal changes to the body e.g. during pregnancy, after menopause or use of birth control products, wearing of unsuitable underwear, during menstruation, diabetes-suffering women, and more. The risk of undesirable spread of the microorganisms from rectum to the urogenital area should be considered as well. All the changes may cause infections resulting in compromising of the natural microbial flora, change to physiological pH value of the vaginal mucosa and external genitals followed by microbial infections that cause vaginitis, urinary tract infection, and common skin inflammations, in particular intertrigo. The infections mentioned above are most often associated with the following families of microorganisms: Escherichia, Enterococcus, Psedomonas, Proteus, Klebsiella, Streptococcus, Staphylococcus, Gardnerella, Ureplasma, Bacteriodes, Peptococcus, Neisseria, Serratia, Corynebacterium, Clostridium, and Candida. The listed specification of the microorganisms narrowly correlates with the fact that there is increased risk of migration of the microorganisms from the rectum to vagina and the areas of external genitals due to shorter distance between the rectum and the urogenital tract. In particular danger are young women without microflora sufficiently developed in the urogenital area so far, and older women already without sufficient protective vaginal microflora that also influences stability of the physiological pH value due to hormonal changes. Increased pH value may result both in bacterial vaginosis or aerobic vaginosis, and is a significant indicator of the abnormal vaginal microflora. Compromising of the microbial balance may have negative impacts on fertility of the women.

Today a lot of known products are commercially available for adjustment or reduction of the pH value of the vaginal environment and external genitals. Said products are based on simple application of creams, gels, and intimate soaps with lactic acid as ingredient being the most important natural factor, which provides acid pH value of the vaginal mucosa and external genitals. Under normal circumstances, Lactobacillus family bacteria produce lactic acid of a healthy woman in particular. However, if the stability of the vaginal microbial flora is compromised, lactic acid production drops in particular due to reduction of lactobacillus, and the mucosa pH value increases accordingly. Subsequent increase of the pH value brings radically increased risk of the inflammation spreading. In particular under these conditions it is appropriate to adjust the increased pH value in a natural way, i.e. with lactic acid in combination with probiotic bacteria by external administration either in the form of an intimate gel, soap, or other preparations. Provided that the probiotic culture is combined with the prebiotic component, so-called symbiotic component is formed under which lactic acid due to reduction of the pH value establishes an optimum environment for propagation of the probiotic culture, and the prebiotic component is also used as a source of selective nutrients for the probiotic culture, whereby propagation of the probiotic bacteria is maximally supported especially in or on a sanitary slip.

At present, commercially available are on the market the products of intimate hygiene such as sanitary towels, daily or night intimate slips, slips, tampons being fortified by lactic acid. UV 23192 discloses fortification of the absorption core by a crystalline form of lactic acid to reduce the pH value of the preparations of in particular feminine intimate hygiene products, being however based on micronizing and quick dissolution of lactic acid, which results in uncontrolled and quick reduction of the pH value of a slip with short-term effect. Also, WO 2008/060200 discloses a sanitary product that contains a mixture for suppression of microbial spreading in urogenital area. The mixture contains extracellular product made from at least one species of the probiotic bacteria, and at least one additive in the form of an organic acid with the pH value lower than 5.5 and/or salt thereof, wherein the extracellular product includes for example antimicrobial peptides and not the probiotic bacteria alone. The acid may be lactic, acetic, ascorbic, citric, and more. Said sanitary product does not deal with application of lactic acid alone, and focuses only on lactobacilli that produce said acid. DE 2309575 also discloses embodiments of sanitary tampons where the tampons are impregnated by lactic acid, glycogen, or sugar or Lactobacillus vaginalis bacteria. All other known patents focus on use of the probiotic bacteria that produce lactic acid. To date, however, there is not a sanitary product on the market with lactic acid applied either on the absorption core of the sanitary product itself or directly on non-woven fabric or toilet paper being in contact with skin and external genitals, with sustained and corrected release through monoglyceryl monoesters coating of the crystalline lactic acid or salts thereof with very fast reduction of the pH value of the used hygienic product to the required physiological pH value of the external genitals, i.e. ideally from 5 to 5.5.

The objection of the invention is to provide a sanitary product with an active ingredient to reduce the pH value of the vaginal environment and/or external genitals, which comprises lactic acid or salts thereof being stabilized effectively in the sanitary product, whereby its treatment and preventive effects are increased, and which maintains the optimum pH value for propagation of the probiotic bacteria. The sanitary product according to the present invention should represent a new hygienic product of which composition naturally contributes to the stabilization of the pH value and microflora of the external genitals, rectum, and skin in private parts.

### SUMMARY OF THE INVENTION

The drawbacks mentioned above are removed by a sanitary product with an active ingredient for reduction of the pH value of vaginal environment and/or external genitals according to the present invention. The active ingredient contains the crystalline lactic acid or salts thereof, and is arranged on a surface layer of the sanitary product designed for contact with body and/or in the inner structure of the sanitary product. According to the present invention, lactic acid and/or salts thereof are contained in the active ingredient in the form of monoglyceryl monoester coating for sustained release of lactic acid from the sanitary product. Coating of lactic acid and/or salts thereof provides an encapsulated form of the active ingredient being effectively stabilized in the sanitary product, and sustained and controlled release of lactic acid for modification of the pH value of the sanitary product is achieved thereby.

In a preferred embodiment the active ingredient includes, in addition, probiotic culture from the Lactobacillus, Bifidobacterium, or Streptococcus family. Presence of these cultures, i.e. lactic acid fermentation bacteria, provides lactic acid, and optimum environment of the sanitary product is achieved thereby.

In addition, the active ingredient preferably includes a prebiotic component from the group comprising of inulin or fructooligosaccharides. The prebiotic component provides support for proper growth and propagation of the probiotic culture of lactic acid fermentation bacteria that sets a balance in the sanitary product.

In a preferred embodiment, lactic acid and/or salts thereof, probiotic culture, and prebiotic components are homogenized in the active ingredient. Preferably, lactic acid and/or salts thereof are in micronized form.

In a preferred embodiment, the sanitary product consists of lower impermeable layer, absorbing core, and upper surface layer made from woven or non-woven fabric. Lactic acid and/or salts thereof, probiotic culture, and prebiotic component, i.e. complete active ingredient has a powder form, and forms a functional layer on reverse side of the upper surface layer.

The functional layer is preferably covered with a sticky layer for combination with the absorbing core. Preferably, the sticky layer consists of a layer of fusible glue. This fixes the active ingredient into the internal structure of the sanitary product, and the latter consists of one complete structure with the functional features described above.

In a preferred embodiment, the sanitary product has a form of a women's sanitary towel or daily or night intimate slip or slip or diaper or incontinence pads or tampon. Thus, the use of the sanitary product is very diverse.

The advantages of the sanitary product with the active ingredient for reduction of the pH value of vaginal environment and/or external genitals according to the present invention consist in particular in that the active ingredient is effectively stabilized in the sanitary product, whereby the treatment and preventive effects of the sanitary product are improved, and which keeps the optimum pH value for propagation of the probiotic bacteria. The sanitary product according to the present invention represents a new hygienic product of which composition naturally contributes to the stabilization of the pH value and microflora of the external genitals, rectum, and skin in private parts.

### DESCRIPTION OF THE DRAWINGS

The invention will be described more in detail in the following figures, where:

FIG. 1 shows a cross-section view of the sanitary product during application of the active ingredient onto the fabric fibres,

FIG. 2 shows a view of the sanitary product in the form of a sanitary towel,

FIG. 3 shows a cross-section view of the sanitary product of FIG. 2,

FIG. 4 shows a view of the sanitary product in the form of a diaper,

FIG. 5 shows a cross-section view of the sanitary product of FIG. 4.

### DETAILED DESCRIPTION OF THE PREFERRED EMBODIMENTS OF THE INVENTION

It is understood that specific embodiments of the invention described below are indicative only and do not in any way limit the embodiments of the invention to these specific shown here. The persons skilled in the art could find or derive under routine experimental procedures either higher or lower number of equivalents to specific embodiments of this invention being described here. These equivalents will be included in the scope of the following claims as well.

### EXAMPLE 1

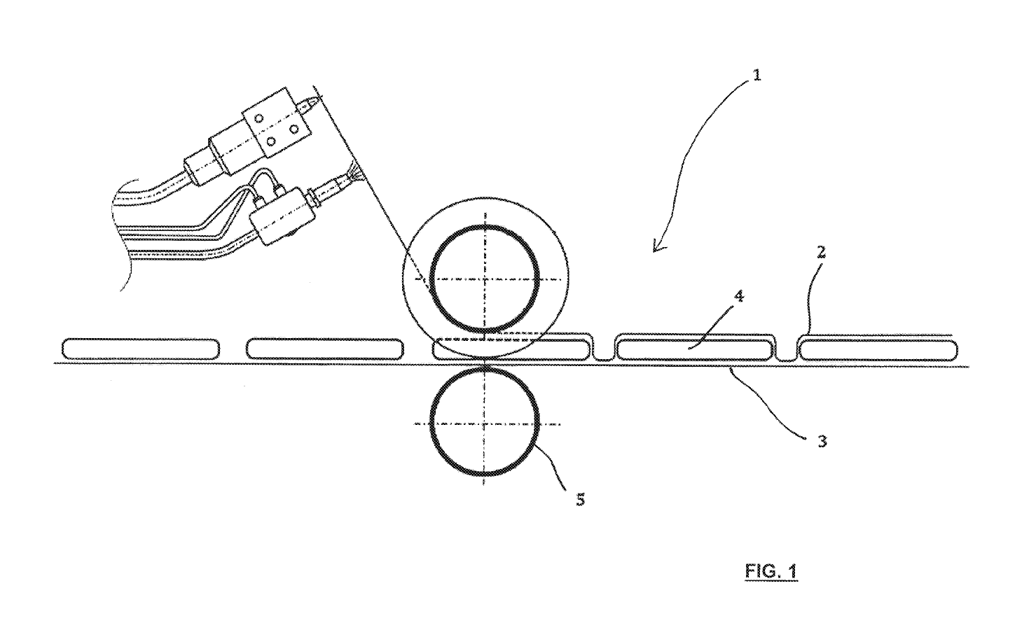

FIG. 1 shows a sanitary product 1 with the active ingredient for reduction of the pH value of the vaginal environment, where 70% wt. crystalline lactic acid is coated with DIMODAN (Danisco) distilled monoglyceride through gentle grinding of agitated mixture in a fragmentation grinder at room temperature without heating in 3 weight ratios 1:2. On the reverse side of the top surface layer 2, i.e. topsheet of a sanitary towel, 0.1 g of powder mixture of the coated lactic acid, probiotic culture Lactobacillus acidophilus is applied, in particular sprayed using dried and cleaned compressed air up to the final concentration 1×10⁸ CFU/g homogenized with 0.05 g of inulin prebiotics. Upon fixing of the powder mixture on the reverse side of the top surface layer 2 of the sanitary tower, said surface layer 2 is provided with homogenous micro-coat of a sticky layer, i.e. the layer 7 of the fusible glue at 3.5 to 5 g/m² being applied using a fixation roll 5. In combination of the upper surface layer 2 made from non-woven fabric with the remaining components of the sanitary towel, the fixation rolls strongly presses the homogenous powder surfaces on the lower impermeable layer 3.

### EXAMPLE 2

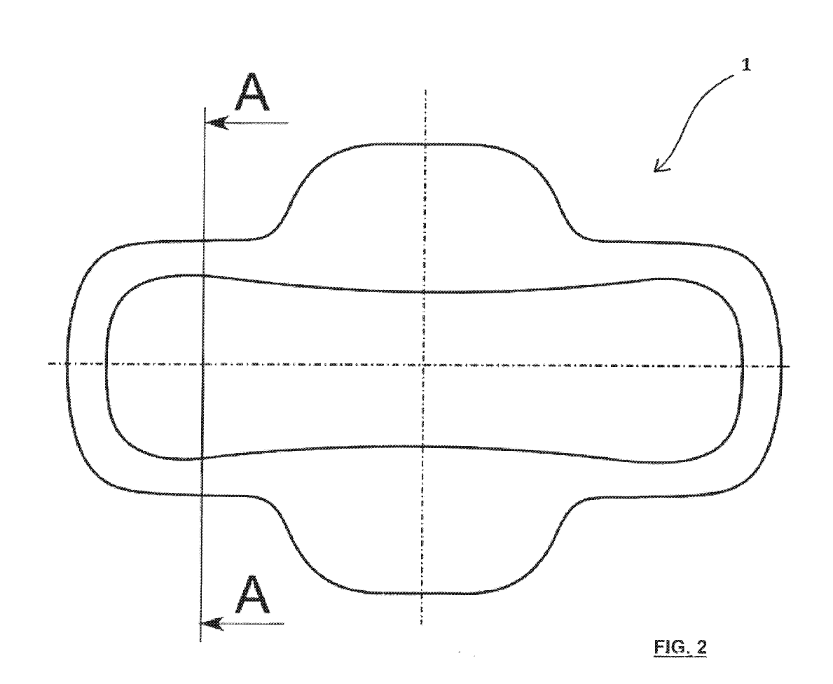

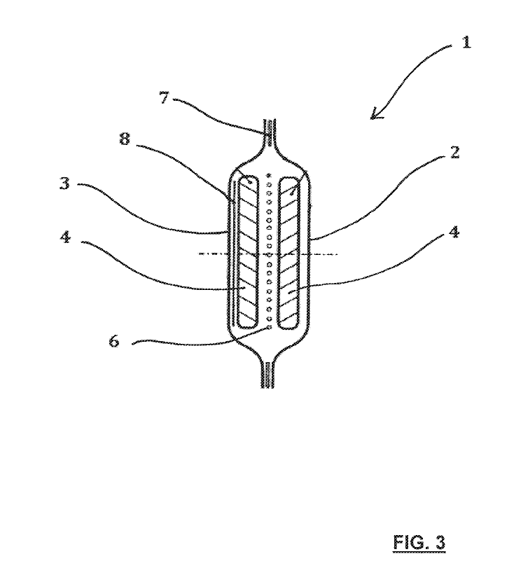

Coating of crystalline lactic acid and calcic salts thereof, i.e. of calcium lactate pentahydrate in 60:40 ratio takes place using monoglyceride alginate in a large-scale extruder. The absorbent forming a part of the absorbing core 4 of the sanitary product 1 is homogenized prior to its application to the absorbing core 4 in the ratio of 80 parts of the absorbent and 19 parts of coated lactic acid with the probiotic culture L. acidophilus up to the final concentration 1×10⁹ CFU, and 1 part of 95% wt. fructooligosaccharide as the prebiotics. The mixture formed in this way is used for production of the absorbing core 4 of the sanitary product 1 in the form of a sanitary towel either sandwiched between two cellulose cores or on surface of the absorbing core 4 before folding, where the sanitary towel described in this example is shown in FIG. 2. FIG. 3 shows a section view of the sanitary towel. Calcium lactate pentahydrate and lactic acid are micronized prior to the application.

### EXAMPLE 3

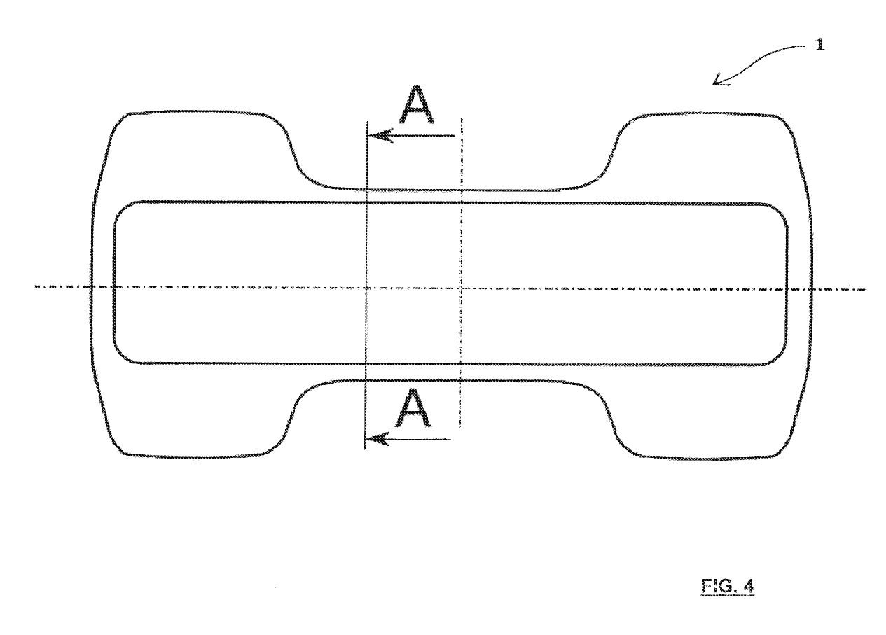

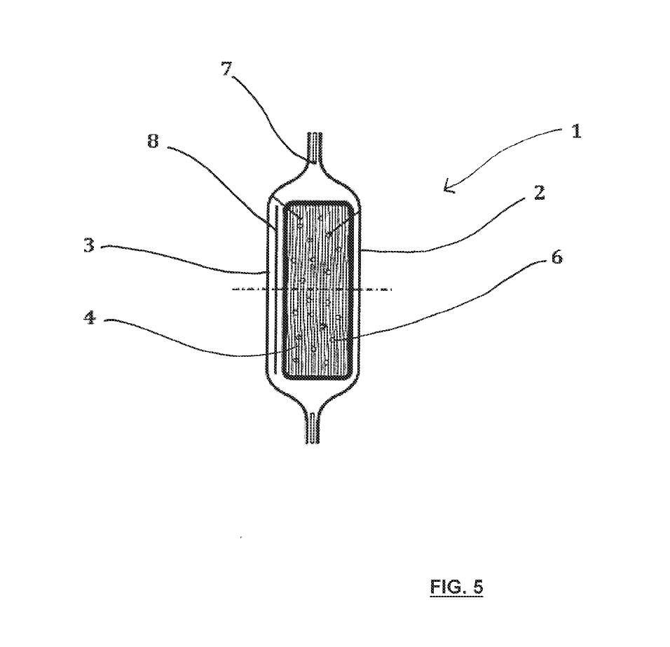

The absorbent forming the absorbing core 4 of the sanitary product 1 in the form of a diaper is homogenized by coated lactic acid prior to application to the absorbing core 4 of the diaper. The coating is a dry process in the fragmentation grinder at 70 revs per minute. The mixture for preparation of coated lactic acid is made from 40 parts of 60% wt. crystalline lactic acid and 60 parts of glyceryl stearate with the pH value adjusted to 5.5. The final superabsorption mixture was obtained by homogenizing in a barrel homogenizer in the ratio of 99 parts of the absorbent and 1 part of coated lactic acid. The mixture prepared in this way is used for application of the coated lactic acid enriched-superabsorbent 6 onto the fiberized cellulose that forms the absorbing core 4 of the diaper as shown in FIG. 4, or applied directly on the upper surface layer 2 of the non-woven fabric (topsheet) in case the absorbing core 4 consists of the superabsorbent 6 and distribution strip 8 only. FIG. 5 shows a section view of the diaper.

### EXAMPLE 4

The absorbent forming the absorbing core 4 of an incontinence pad is homogenized by glyceryl lactate-coated crystalline lactic acid prior to application to the absorbing core 4. The mixture prepared in this way is used for production of the absorbing core 4, wherein lactic acid is micronized prior to the application.

### INDUSTRIAL APPLICABILITY

The sanitary product with an active ingredient for reduction of the pH value of vaginal environment and/or external genitals according to this invention can be in particular applied in women's hygiene in the form of sanitary towels, daily and night intimate slips, slips, and tampons used both during the menstruation cycle as well as between the menstruation cycles to provide quality women's intimate hygiene in order either to stabilize or support recovery of physiological pH value by sustained and gentle release of lactic acid, and recovery of natural microflora of vagina and external genitals, skin, and rectum by addition of the probiotic culture with prebiotics used as a selective energy source. Furthermore, the sanitary product can be used in routine children's hygiene in the form of diapers, as well as incontinence pads, where the pH value reduction limits propagation of pathogenic microorganisms and reduces the risk of surface inflammations and intertrigo.

1. A sanitary product with an active ingredient for reduction of the pH value of vaginal environment and/or external genitals, where the active ingredient contains crystalline lactic acid or salts thereof, and is applied on the surface layer of the sanitary product designed for contact with body and/or in the internal structure of the sanitary product wherein lactic acid and/or salts thereof are contained in the active ingredient in the form of monoglyceryl monoester coated lactic acid for sustained release of lactic acid from the sanitary product.

2. The sanitary product according to claim 1, the active ingredient includes, in addition, probiotic culture from the Lactobacillus, Byidobacterium, or Streptococcus family.

3. The sanitary product according to 2, wherein the active ingredient further contains prebiotic component from the group consisting of inulin or fructooligosaccharides.

4. The sanitary product according to claim 1, wherein lactic acid and/or salts thereof, probiotic culture, and prebiotic components are homogenized in the active ingredient.

5. The sanitary product according to claim 1, wherein lactic acid and/or salts thereof are micronized.

6. The sanitary product according to claim 1, wherein it consists of lower impermeable layer, absorbing core, and upper surface layer made from woven or non-woven fabric, wherein the active ingredient has a powder form and forms the functional layer on the reverse side of the upper surface layer.

7. The sanitary product according to claim 6, wherein the functional layer is covered by a sticky layer for combination with the absorbing cover.

8. The sanitary product according to claim 7, wherein the sticky layer consists of the layer of fusible glue.

9. The sanitary product according to claim 1, wherein the sanitary product has a form of a women's sanitary towel or daily or night intimate slip or slip or diaper or incontinence pads or tampon.
